(19) United States
(12) Patent Application Publication
(10) Pub. No.: US 2024/0165060 A1
(43) Pub. Date: May 23, 2024

(54) METHOD AND COMPOSITION FOR LOWERING TOTAL SYSTEMIC CHOLESTROL, LDL CHOLESTEROL, AND NON-HDL CHOLESTEROL

(71) Applicant: BioKier, Inc., Chapel Hill, NC (US)

(72) Inventor: Jerzy Ryszard Szewczyk, Chapel Hill, NC (US)

(73) Assignee: BioKier, Inc., Chapel Hill, NC (US)

(21) Appl. No.: 18/430,750

(22) Filed: Feb. 2, 2024

Related U.S. Application Data
(63) Continuation-in-part of application No. 17/554,076, filed on Dec. 17, 2021.

(51) Int. Cl. A61K 31/19; A61K 9/00; A61K 9/20; A61K 9/28; A61K 31/366; A61K 31/40; A61K 31/455; A61P 3/06

(52) U.S. Cl. CPC A61K 31/19; A61K 9/0053; A61K 9/2013; A61K 9/2846; A61K 9/286; A61K 31/366; A61K 31/40; A61K 31/455; A61P 3/06

(57) ABSTRACT

The present invention relates to a method and composition of treating a lipid related disease such as diabetes, obesity, hypercholesterolemia, and the like by delivery of butyrate directly to the colon by bypassing the upper digestive tract in combination with a second composition that lowers cholesterol, LDL cholesterol and non-LDL cholesterol.

This application is a Continuation in Part of application Ser. No. 17/554,076 filed on Dec. 17, 2021 and which is referenced here in its entirety.

### STATEMENT REGARDING FEDERALLY SPONSORED RESEARCH OR DEVELOPMENT

This invention was partially funded by the National Institutes of Health, government contract number 2R44DK107080-04.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a novel method and composition for treating a lipid related disease by lowering at least one of total systemic cholesterol, low-density lipoproteins (LDL) cholesterol, and non-high-density lipoprotein (non-HDL) cholesterol. In particular, the present invention relates to the lowering of at least one of total systemic cholesterol, low-density lipoproteins (LDL) cholesterol, and non-high-density lipoprotein (non-HDL) cholesterol by delivery of butyric acid and a cholesterol lowering composition to release essentially entirely in the colon.

### Description of Related Art

Cholesterol is present in every cell of the body and has important natural functions when it comes to digesting foods, producing hormones, and generating vitamin D. The body produces it, but it is also consumed through food intake. It is waxy and fat-like in appearance. There are two types of cholesterol: low-density lipoproteins (LDL), or “bad” cholesterol and high-density lipoproteins (HDL), or “good” cholesterol.

Cholesterol has four primary functions, without which we could not survive. These functions are: contributing to the structure of cell walls, making up digestive bile acids in the intestine, allowing the body to produce vitamin D, and enabling the body to make certain hormones. High total systemic cholesterol is a significant risk factor for coronary heart disease and a cause of heart attacks. A build-up of cholesterol is part of the process that narrows arteries, called atherosclerosis. In atherosclerosis, plaques form and cause restriction of blood flow. Reducing the intake of fat in the diet is one way to manage total systemic cholesterol levels.

Several drugs are in current use for treating a lipid related disease by lowering at least one of total systemic cholesterol, LDL, and non-HDL cholesterol in humans, including statins, PCSK9 inhibitors, fibric acid derivatives (also called fibrates), bile acid sequestrants (also called bile acid resins), nicotinic acid (also called niacin), selective cholesterol absorption inhibitors, omega-3 fatty acids and fatty acid esters, and adenosine triphosphate-citrate lyase (ACL) inhibitors. Because these current treatments have several limitations associated with them, there is still a need for alternative therapies to lower total systemic cholesterol, LDL, and non-HDL cholesterol.

There are a number of compositions designed to deliver a medicament to the lower gut bypassing the stomach and upper intestine. One in particular are the three-component matrix structures, such as disclosed in U.S. Pat. No. 7,431,943 issued Oct. 7, 2008 to Villa et al. and is incorporated herein in its entirety by reference. Another method of delivering drugs to the lower intestine is described in U.S. patent application Ser. No. 16/805,080, filed Feb. 28, 2020 in the name of Szewczyk et al.

A number of different other formulations are available for delivery of desired compositions to the lower gut including amylose coated tablets, enterically coated chitosan tablets, matrix within matrix or multi-matrix systems, or polysaccharide coated tablets. One example of multi-matrix-controlled release systems is disclosed in U.S. Pat. No. 7,431,943 issued Oct. 7, 2008 to Villa et al. and is incorporated herein by reference. Disclosed is a matrix within matrix design wherein a lipophilic phase and amphiphilic phase are incorporated within the inner matrix and at least a portion of the active ingredient is incorporated into the amphiphilic phase. Others include, for example, liquid-fill hard capsules composed of one or more components which erode slowly in a colonic environment and are coated by a layer composed of materials, which disintegrate upon (close to) entry to the colon. The prime decision is knowing if or why a colon targeted delivery system is better than a non-colon targeted delivery system, since it is assumed, they are equivalent.

### BRIEF SUMMARY OF THE INVENTION

The present invention relates to the discovery that certain butyrate compositions, with other cholesterol lowering compositions, if delivered to the colon bypassing the stomach and upper digestive system (duodenum, jejunum, and ileum), treats a lipid related disease by synergistically decreasing at least one of total systemic cholesterol, LDL cholesterol, and non-HDL cholesterol in humans.

Therefore, in one embodiment, the present invention is a method of treating a lipid related disease by lowering total systemic cholesterol, LDL cholesterol and non-LDL cholesterol comprising:
a) selecting a composition for oral administration, the composition formulated to release butyric acid in a colon targeted delivery system, bypassing the stomach, duodenum, jejunum, and ileum and releasing in the colon and providing a daily dose of butyrate in single or multiple doses comprising 21 mg to 4200 mg; and
b) administering an effective amount of a second composition to the colon of the human sufficient to lower at least one of total systemic cholesterol, LDL cholesterol, and non-HDL cholesterol.

In another embodiment, there is a composition for treating a lipid related disease by lowering at least one of total systemic cholesterol, LDL cholesterol, and non-HDL cholesterol in a human comprising:
a) a pharmaceutical composition for oral administration comprising butyric acid and at least one additional composition that lowers at least one of total systemic cholesterol, LDL cholesterol, and non-HDL cholesterol, the composition formulated to release 21 mg to 4200 mg of butyric acid in a single or multiple dose in a colon targeted delivery system, bypassing the stomach, duodenum, jejunum, and ileum and only releasing in the colon; and
b) an effective amount of a second composition to the colon of the human sufficient to lower at least one of total systemic cholesterol, LDL cholesterol, and non-HDL cholesterol.

### DETAILED DESCRIPTION OF THE INVENTION

While this invention is susceptible to embodiment in many different forms, there will herein be described in detail specific embodiments, with the understanding that the present disclosure of such embodiments is to be considered as an example of the principles and not intended to limit the invention to the specific embodiments shown and described. This detailed description defines the meaning of the terms used herein and specifically describes embodiments in order for those skilled in the art to practice the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps, or acts are in some way inherently mutually exclusive.

The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “treating” refers to alleviating the specified condition, eliminating, or reducing the symptoms of the condition, slowing or eliminating the progression of the condition, and preventing or delaying the initial occurrence of the condition in a subject, or reoccurrence of the condition in a previously afflicted subject.

As used herein, the term “condition or disorder” refers to any condition characterized by total systemic high cholesterol, high LDL cholesterol, or high non-HDL cholesterol in a mammalian subject. Included are the disease states noted herein. In one embodiment, the mammal is a human.

Conventional stomach delivered butyric acid formulations produce dose limiting adverse gastrointestinal issues including diarrhea, nausea, vomiting, dizziness, headaches, and dyspepsia. Frequently, administration must be titrated upwardly in an attempt to accommodate the breakdown in the stomach, however, they just don't appear to work at all. Extended-release formulations have been developed which attempt to deal with this issue, but still do not fully address these complications.

As used herein, a “compound” of the present invention includes all compounds described herein.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art, such as x-ray diffraction patterns, solubility, and melting point.

Certain of the compounds, described herein, contain one or more chiral centers, or may otherwise be capable of existing as multiple stereoisomers. The scope of the present invention includes mixtures of stereoisomers, as well as purified enantiomers or enantiomerically/diastereomerically enriched mixtures. Also, included within the scope of the present invention are the individual isomers of the compounds, as well as any wholly or partially equilibrated mixtures thereof. The present invention also includes the individual isomers of the compounds represented by the formulas above as mixtures with isomers, thereof, in which one or more chiral centers are inverted.

Typically, but not absolutely, the compounds herein include the salts of the present compositions and include the pharmaceutically acceptable salts. Salts encompassed within the term “pharmaceutically acceptable salts” refer to non-toxic salts of the compounds of this invention. Salts of the compounds of the present invention may include acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, thethiodide, thmethylammonium, and valerate salts. Other salts, which are not pharmaceutically acceptable, may be useful in the preparation of compounds of this invention, and these should be considered to form a further aspect of the invention.

The “administering” of a composition of the present invention can refer to oral administration. As described elsewhere herein, the compounds are so formulated to be taken orally so they bypass the upper digestive tract and stomach or taken rectally to deliver the composition to the colon, i.e., large intestine.

As used herein, the term “effective amount” means that amount of a drug or pharmaceutical agent that will elicit the biological or medical response of a tissue, system, animal, or human that is being sought, for instance, by a researcher or clinician. It has been discovered that butyrate lowers total systemic high cholesterol, high LDL cholesterol, and non-HDL cholesterol, but only when it is delivered directly to the colon, while administration orally to the stomach or upper intestine is ineffective.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes, within its scope, amounts effective to enhance normal physiological function. A therapeutically effective amount will produce a “therapeutic effect”.

For use in therapy, therapeutically effective amounts of a compound of the present invention, as well as salts thereof, are presented as a pharmaceutical composition formulated to release in a colon targeted delivery system which bypasses the stomach and upper intestine.

The present invention provides pharmaceutical compositions that include effective amounts of a compound, as herein described, or a salt thereof, and one or more pharmaceutically acceptable carriers, diluents, or excipients. The carrier(s), diluent(s), or excipient(s) must be acceptable, in the sense of being compatible with the other ingredients of the formulation and not deleterious to the recipient of the pharmaceutical composition, as well as consistent with the mode of administration, i.e., oral or rectal.

In accordance with another aspect of the present invention, there is also a process provided for the preparation of a pharmaceutical formulation, including admixing a compound of the present invention or salts thereof, with one or more pharmaceutically acceptable carriers, diluents, or excipients for delivery directly to the colon.

A therapeutically effective amount of a compound of the present invention will depend upon a number of factors. For example, the species, age and weight of the recipient, the precise condition requiring treatment and its severity, the nature of the formulation, and the type of colon targeted delivery system selected are all factors to be considered. The therapeutically effective amount ultimately should be at the discretion of the attendant, physician, or veterinarian. Regardless, an effective amount of a compound of the present invention for the treatment of humans suffering from total systemic high cholesterol, high LDL cholesterol, and high non-HDL cholesterol, generally, should be in the range of 0.01 mg/kg to 100 mg/kg body weight of recipient (mammal) per day. More often, the effective amount should be in the range of 0.3 mg/kg to 60 mg/kg body weight per day. Thus, for a 70 kg adult mammal, the actual amount per day would usually be from 21 mg to 4200 mg. In one embodiment, the amount is 3,000 mg. This amount may be given in a single dose per day or in a number (such as two, three, four, five, or more) of sub-doses per day, such that the total daily dose is the same. An effective amount of a salt or solvate thereof, may be determined as a proportion of the effective amount of the compound of the present invention per se. Similar dosages should be appropriate for treatment of the other conditions referred to herein.

Pharmaceutical formulations may be presented in unit dose forms containing a predetermined amount of an active ingredient per unit dose. Such a unit may contain, as a non-limiting example, 0.5 mg to 1 g of a compound of the present invention, depending on the condition being treated, the route of administration, and the age, weight, and condition of the recipient. Preferred unit dosage formulations are those containing a daily dose or sub-dose, as herein above recited, or an appropriate fraction thereof, of an active ingredient. Such pharmaceutical formulations may be prepared by any of the methods well known in the pharmaceutical art.

The compounds of the present invention, or a salt thereof, are administered by a targeted drug delivery system. In one embodiment, the delivery systems may be employed for targeting drug delivery to the colon and bypassing the upper digestive system and stomach. Such drug delivery systems include, but are not limited to, covalent linkage compositions, polymer coated compositions, compositions embedded in matrices, time-released compositions, redox-sensitive polymer compositions, bioadhesive compositions, micropartical coating compositions, and osmotic delivery compositions. Suitable compositions include those containing polysaccharides, such as chitosan, pectin, chondroitin sulphate, cyclodexthn, dextrans, guar gum, inulin, amylose, and locust bean gum. The compounds may also be coupled with soluble polymers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels. Those of particular effectiveness in the present invention include embodiments comprising sustained-release core of tablet or capsule coated with single or double layer of colon sensitive material, Phloral® (from Intract Pharma, Ltd.) or Phloral® with additional layer separating Phloral® from content of the tablet systems. Phloral® is a dual action (pH and enzymatic activity of the microbiota) product for delivery to the colon. Those skilled in the art will appreciate the use of such compositions for the purposes of targeting delivery of the compounds of the present invention, or a salt thereof, to the colon of the recipient being treated. The methods for the formulation of such compositions for targeted delivery are within the skill in the art, in view of this disclosure.

The compounds of the present invention or a salt thereof may be employed alone or in combination with other therapeutic agents. The compound(s) of the present invention and the other pharmaceutically active agent(s) are administered together or at the same time. The amounts of the compound(s) of the present invention and the other pharmaceutically active agent(s) involve the relative timings of administration which will be selected in order to achieve the desired combined therapeutic effect. The administration in combination with a compound of the present invention or a salt or solvate thereof with other treatment agents may be in combination by administration concomitantly in: (1) a unitary pharmaceutical composition, including both compounds; or (2) separate pharmaceutical compositions, each including one of the compounds. Alternatively, the combination may be administered separately in a sequential manner, wherein one treatment agent is administered first and the other second, or vice versa. Such sequential administration should be close in time or remote in time.

The compounds of the present invention may be used in the treatment of a variety of disorders and conditions. As such, the compounds of the present invention may be used in combination with a variety of other therapeutic agents useful in the treatment of those disorders or conditions. Included are statins, niacin, 2-Azetidinones, bile acid sequestrants, and PCSK9 inhibitors.

### EXAMPLES

### Example 1

Sodium butyrate, 100-1,000 mg, is mixed with several excipients and pressed into the tablet. Excipients are selected to assure sustained-release of butyrate (2-10 hrs) in the colon environment. The tablet is coated with Phloral® or with an additional layer separating Phloral® from the core of the tablet and Phloral®. This coating reacts (is digested) to pH and enzymes to deliver to the colon.

### Example 2

The tablets of colon-targeted butyrate formulation (500 mg per tablet) were dosed to 14 diabetic subjects with other comorbidities over a 4-week period. Total, LDL, and HDL cholesterol were measured in addition to other biomarkers at the beginning and the end of the treatment period.

| | Total | | LDL | | non-HDL | | Chol | Duration |
|---|---|---|---|---|---|---|---|---|
| Subject | Before | After | Before | After | Before | After | Meds | on statins |
| 401 | 140 | 135 | 85 | 72 | 101 | 100 | Lipitor, | 11 Y |
| | | | | | | | Niacin | |
| 402 | 185 | 182 | 112 | 108 | 152 | 151 | | |
| 403 | 198 | 148 | 111 | 71 | 154 | 117 | Lipitor, | 7 Y |
| | | | | | | | Niacin | |
| 404 | 160 | 141 | 48 | 55 | 107 | 97 | | |
| 405 | 123 | 133 | 68 | 59 | 87 | 103 | Zocor | 17 Y |
| 406 | 176 | 131 | 101 | 69 | 115 | 79 | Lipitor | 4 Y |
| 407 | 142 | 108 | 77 | 43 | 101 | 71 | Lipitor | 6 Y |
| 408 | 197 | 188 | 100 | 106 | 134 | 124 | | |
| 409 | 234 | 165 | 143 | 97 | 169 | 123 | | |
| 410 | 203 | 177 | 105 | 106 | 157 | 136 | | |
| 411 | 176 | 148 | 106 | 86 | 126 | 108 | Zocor | 11 Y |
| 412 | 228 | 199 | 141 | 98 | 175 | 157 | | |
| 413 | 161 | 143 | 98 | 89 | 117 | 109 | Lipitor | 7 Y |
| 414 | 131 | 125 | 48 | 51 | 83 | 83 | Lipitor | 4 Y |
| Average | 175 | 152 | 96 | 79 | 127 | 111 | | |
| SD | 34 | 26 | 28 | 22 | 30 | 25 | | |
| p | | 0.0012 | | 0.0081 | | 0.0036 | | |

Table 1: The baseline and after 4 weeks of treatment with colon-targeted butyrate values of total, HDL, LDL, and non-HDL cholesterol. The lowering of total LDL and non-HDL cholesterol were significantly lower after the treatment despite the fact that some subjects in the trial had very low initial LDL (404 and 414). The effect of colonic butyrate was evident in both statin-treated and naïve subjects. Additionally, several patients in which LDL did not change were only borderline hypercholesterolemic.

### Example 3

Sodium butyrate, 100-500 mg, is mixed with 100-500 mg of glutamine and several excipients and pressed into the tablet. Excipients are selected to assure sustained release of butyrate+glutamine (2-10 hrs) in the colon environment. The tablet is coated with Phloral® or with an additional layer separating Phloral® from the core of the tablet and Phloral®. This coating reacts to pH and enzymes to deliver to the colon.

### Example 4

Sodium butyrate, 100-500 mg, is mixed with 100-500 mg of TGR-5 agonist and several excipients and pressed into the tablet. Excipients are selected to assure sustained release of butyrate+TGR5 agonist (2-10 hrs) in the colon environment. The tablet is coated with Phloral® or with an additional layer separating Phloral® from the core of the tablet and Phloral®. This coating reacts to pH and enzymes to deliver to the colon.

### Example 5

Sodium butyrate, 100-500 mg, is mixed with 100-500 mg of niacin and several excipients and pressed into the tablet. Excipients are selected to assure sustained release of butyrate+niacin (2-10 hrs) in the colon environment. The tablet is coated with Phloral® or with an additional layer separating Phloral® from the core of the tablet and Phloral®. This coating reacts to pH and enzymes to deliver to the colon.

### Example 6

Sodium butyrate, 100-500 mg, is mixed with 100-500 mg of GPR-41 and/or GPR-43 agonist and several excipients and pressed into the tablet. Excipients are selected to assure sustained release of butyrate+GPR-41-or 43 agonist (2-10 hrs) in the colon environment. The tablet is coated with Phloral® or with an additional layer separating Phloral® from the core of the tablet and Phloral®. This coating reacts to pH and enzymes to deliver to the colon.

What is claimed is:

1. A method of treating a lipid related disease by lowering total systemic cholesterol, LDL cholesterol and non-LDL cholesterol comprising:
a) selecting a composition for oral administration comprising, the composition formulated to release butyric acid in a colon targeted delivery system, bypassing the stomach, duodenum, jejunum, and ileum and releasing in the colon and providing a daily dose of butyrate in single or multiple doses comprising 21 mg to 4200 mg; and
b) administering an effective amount of a second composition to the colon of the human sufficient to lower at least one of total systemic cholesterol, LDL cholesterol, and non-HDL cholesterol.

2. The method according to claim 1 wherein the colon targeted delivery system comprises:
a) a butyrate core;
b) the butyrate core coated with a neutral polymer in a thickness from about 5-100 microns; and
c) the neutral polymer coated with a composition that only dissolves in the colon when the tablet is given orally.

3. The method according to claim 1 wherein the composition has a dual-trigger colon-targeting coating.

4. The method according to claim 1, wherein the neutral polymer layer has a thickness of: from about 10 to 50 microns.

5. The method according to claim 1, wherein the butyrate core is further comprised of one or more additional medically active compounds.

6. The method according to claim 5, wherein the additional medically active compounds is a statin.

7. The method according to claim 5, wherein the additional medically active compound is niacin.

8. The method of lowering at least one of total systemic cholesterol, LDL cholesterol, and non-HDL cholesterol in a human according to claim 5, wherein the additional medically active compound is selected from the group consisting of 2-azetidinones, bile acid sequestrants, and PCSK9 inhibitors.

9. The method according to claim 1 wherein the second composition is formulated to release the second composition in a colon targeted delivery system either in combination with butyrate or separately.

10. A composition for treating a lipid related disease by lowering at least one of total systemic cholesterol, LDL cholesterol, and non-HDL cholesterol in a human comprising:
a) a pharmaceutical composition for oral administration comprising butyric acid and at least one additional composition that lowers at least one of total systemic cholesterol, LDL cholesterol, and non-HDL cholesterol, the composition formulated to release 21 mg to 4200 mg of butyric acid in a single or multiple dose in a colon targeted delivery system, bypassing the stomach, duodenum, jejunum, and ileum and only releasing in the colon; and
b) an effective amount of a second composition to the colon of the human sufficient to lower at least one of total systemic cholesterol, LDL cholesterol, and non-HDL cholesterol.

11. The composition according to claim 10, wherein the at least one additional composition is a statin composition.

12. The composition according to claim 10, wherein the at least one additional composition is niacin.

13. The composition according to claim 10, wherein the at least one additional composition is selected from the group consisting of glutamine, bile acids or their analogs and an L-cell stimulating compound.

14. The composition according to claim 10 wherein the second composition is formulated to release the second composition in a colon targeted delivery system either separately or together with butyric acid.
